(19) United States
(12) Patent Application Publication
(10) Pub. No.: US 2013/0216589 A1
(43) Pub. Date: Aug. 22, 2013

(54) ORAL VACCINE FOR AQUATIC ANIMALS

(71) Applicants: Huey-Lang Yang, Tenafly, NJ (US); Yi-Min Chen, Tainan (TW); Han-You Lin, Tainan (TW)

(72) Inventors: Huey-Lang Yang, Tenafly, NJ (US); Yi-Min Chen, Tainan (TW); Han-You Lin, Tainan (TW)

(73) Assignee: Rich Content Biotech INC., Taipei City (TW)

(21) Appl. No.: 13/399,161

(22) Filed: Feb. 17, 2012

(51) Int. Cl. A61K 39/12; A61K 9/00; A61P 37/04; A61K 39/02

(52) U.S. Cl. 424/400; 424/234.1; 424/235.1; 424/204.1

(57) ABSTRACT

The preset invention relates to an oral vaccine with an enhanced protective efficacy for aquatic animals comprising an antigen, which is expressed in Listonella sp., such as L. anguillarum, as an expression host transformed with an expression vector comprising the nucleic acid encoding the antigen, such as a viral subunit.

### FIELD OF THE INVENTION

The present invention relates to an oral vaccine for aquatic animals, particularly an oral vaccine with an enhanced immune response.

### BACKGROUND OF THE INVENTION

Oral vaccine containing a viral antigen is the most desirable immunization method to prevent fish disease at early larvae and fingerling stage, as it is easy to use, safe and stress-free for fish [Campbell R et al., Uptake of Vibrio anguillarum vaccine by Artemia salina as a potential oral delivery system to fish fry. Fish Shellfish Immunol. 3:451-459, 1993; and Vervarcke S et al., Oral vaccination of African catfish with Vibrio anguillarum O2: effect on antigen uptake and immune response by absorption enhancers in lag time coated pellets. Fish Shellfish Immunol. 16:407-414, 2004]. For example, fish larvae are developed in water environment that contains pathogens, also encounters diseases, and thus oral vaccine encapsulated in rotifer or Artemia was developed as the natural starting feed to enable the early and easy immunization. However, fish oral vaccines were known to have low efficacy and thus the potency needed to be bettered [Shao Z J; Aquaculture pharmaceuticals and biologicals: current perspectives and future possibilities. Adv Drug Deliv Rev. 50:229-243, 2001; and Lin C-C et al.; An oral nervous necrosis virus vaccine that induces protective immunity in larvae of grouper (Epinephelus coioides). Aquaculture 268:265-273, 2007].

Conventionally, adjuvant, either a chemical composition or a physical structure, that can enhance efficacy of a particular vaccine rare used in injective vaccine. Oral vaccine often cannot use chemical based adjuvant, as chemical based adjuvant will cause side effect, especially, deformity in fish larva that is still at the embryonic stage. A biological based adjuvant that can be digested and with no side effect would be preferred for a oral vaccine.

To develop aquaculture industry, a method to enhance the efficacy of the oral vaccine is desirable.

### SUMMARY OF THE INVENTION

The present invention relates to a new approach to enhance the protective efficacy of an oral vaccine for aquatic animals by expressing a specific antigen in a bacterial expression host that has activity to enhance innate immunity and confers adjuvant function, and used to construct an oral vaccine [Lin C-C et al.; An oral nervous necrosis virus vaccine that induces protective immunity in larvae of grouper (Epinephelus coioides). Aquaculture 268:265-273, 2007]. It is found in the invention that the prophylactic and therapeutic efficacy of the antigen contained in an oral vaccine if Listonella sp., such as L. anguillarum, is used as an expression host for the antigen.

In one aspect, the invention provides an oral vaccine with an enhanced protective efficacy for aquatic animals comprising an antigen, which is expressed in Listonella sp., such as L. anguillarum, as an expression host transformed with an expression vector comprising a specific nucleic acid sequence encoding the antigen, such as a viral subunit.

In another aspect, the invention provides an oral vaccine with an enhanced protective efficacy for aquatic animals comprising an antigen, which is expressed in L. anguillarum as an expression host transformed with an expression vector comprising the antigen, and then is encapsulated in rotifer sp., such as Brachionus plicatilis.

In another aspect, the invention provides an oral vaccine with an enhanced protective efficacy for aquatic animals comprising an antigen, which is expressed in L. anguillarum as an expression host transformed with an expression vector comprising the antigen, and then is encapsulated in Artemia sp., such as Artemia nauplii.

In the other aspect, the invention provides a method for preparing the oral vaccine preparation to have concurrent adjuvant activity, the invention.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments, which are presently preferred. It should be understood, however, that the invention is not limited to the embodiments shown.

Selecting a bacterium that when inactivated can stimulate innate immunity and function as adjuvant for vaccine preparation

Selection of shuttle promoter that can express in the bacterium

Construct a recombinant plasmid by fusing gene or part of gene of a specific target antigen onto the promoter, as exampled in the drawings:

FIG. 1 shows the construction of a shuttle vector that can be genetic manipulated in E. coli and then transform into expression host. Vector p groEp-NNVcp fusion plasmid according to the invention.

FIG. 2 provides the DNA sequence of groE promoter region from nucleotide numbers −541 to +58 relative to translation initiation site (ATG underlined).

FIG. 3 provides the NNVcp production of recombinant L. anguillarum under different induction temperatures (M: molecular marker; solid arrow, NNVcp).

FIG. 4 shows the NNVcp production in the two subunit vaccines by using SDS-PAGE (A), western blot (B), and ImageJ software (C) (M: molecular marker; Lane 1: Mock E. coli; Lane 2: E. coli-based subunit vaccine; Lane 3: Mock L. anguillarum; Lane 4: L. anguillarum-based subunit vaccine. Solid arrow, NNVcp; Dash line, aggregrated NNVcp).

FIG. 5 shows the standard curves used to determine the amount of (A) E. coli, and (B) L. anguillarum encapsulated in each Artemia.

FIG. 6 shows the increase of antibody titers against NNV coat protein in groupers 7 days after oral vaccination by L. anguillarum-based oral subunit over prior E. coli based oral NNV vaccine (P<0.05).

FIG. 7 shows the time course of expression levels of three innate immunity-related genes, TNF-α(A), IL-1β(B), Mx in viscera (C); and Mx in brain (D), after oral consumption of E. coli or V. anguillarum (*: P<0.05) to demonstrate the enhancement of innate immunity by this L. anguillarum-based vaccine.

FIG. 8 shows the cumulative mortalities of groupers after challenge with NNV (◯: Placebo, challenged with L15 medium indicating the adjuvant activity of L. anguillarum-based vaccine in protective immunity. Δ: Placebo, challenge with NNV; +: Mock L. anguillarum; X: Mock E. coli; ♦: L. anguillarum-based vaccine : E. coli-based vaccine).

FIG. 9 shows the oral vaccine construction, in general, a specific nucleic acid sequence coding for a specific antigen was cloned and expressed in a bacterial host, then the recombinant bacteria was uptake through filtration by a natural multiple cell animal that is a natural starting feed of feed larva. This has been invented by Yang et a.l and were patented in U.S. Pat. No. 6,872,386 B2, ORAL VACCINES. Mar. 29, 2005 and U.S. Pat. No. 7,807,144 B2 Oct. 5, 2010

### DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

As used herein, the term “vaccine” refers to a biological preparation that induces immunity to a particular disease in a subject, which typically contains an agent that resembles a disease-causing microorganism, and is often made from weakened or killed forms of the microbe, its toxins or one of its surface proteins, and can stimulate the immune system to recognize the agent as foreign, destroy it and remember it so that the immune system can more easily recognize and destroy any of these microorganisms that infect the subject later. In the invention, the vaccine is prophylactic and/or therapeutic.

As used herein, the term “aquatic animal” refers to either vertebrate or invertebrate living in water for most or all of its life; particularly fishes or shrimps.

As used herein, the term “natural starting feed” refers to multiple cell animals living in water and usually are food for fish or shrimp larvae or fingerlings; particularly Artemia or Rotifers.

As used herein, the term “promoter” refers to a regulatory region of DNA located upstream of a gene, providing a control point for regulated gene transcription in a host. In the invention, the promoter includes any regulatory region for expression of an antigen in a Listonella host.

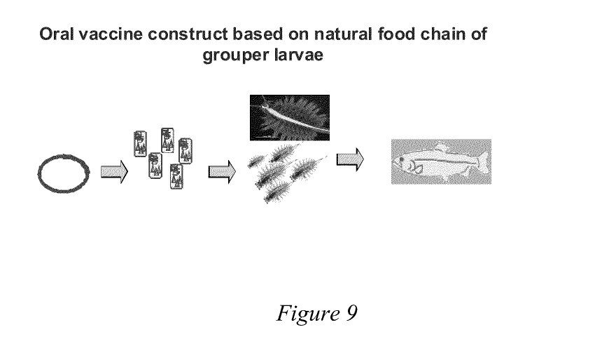

According to the invention, an oral vaccine with an enhanced protective efficacy for aquatic animals is provided. The oral vaccine comprises an antigen, which is expressed in Listonella sp., such as L. anguillarum, as an expression host transformed with an expression vector comprising the nuclei acid encoding the antigen as illustrated in FIG. 9.

The present invention relates to an oral vaccine of the invention is either for prophylactic vaccination or for therapeutic vaccination. In the invention, the antigen used in the oral vaccine may be any agent resembles a disease-causing microorganism, and is often made from weakened or killed forms of the antigen. One example of the antigens in the invention is a foreign polypeptide of interest. In one example, the antigen is a viral subunit. The viral subunit includes non-recombinant or recombinant viral subunit. Recombinant viral subunits are usually used for preparing oral vaccine for aquatic animals. In one example of the invention, the antigen is a subunit of Nervous Necrosis Virus (NNV).

According to the invention, the invention is characterized by using Listonella sp. (formerly Vibrio sp.) as an expression host, providing an unexpectedly prophylactic and therapeutic efficacy against infection. In one example of the invention, Listonella anguillarum was used as an expression host to prepare an oral vaccine for aquatic animals, providing an improved protective efficacy of the oral vaccine.

The oral vaccine may include a multiple-cell organism that has fed on, and therefore contains, a single-cell organism. The single-cell organism fed to the multiple-cell organism has been transformed to express a recombinant antigen that can induce an immune response in the aquatic animal. The just-described multiple-cell organism, fed to the aquatic animal, serves as an oral vaccine to the animal. In other words, an antigen, being expressed in a single-cell, multiple-cell organism or in a aquatic animal, is delivered to the aquatic animal via two steps of feeding, i.e., the above-described single-cell organism fed to the multiple-cell organism, and the multiple-cell organism from previous step fed to the aquatic animal. This has been invented by Yang et a.l and were patented in U.S. Pat. No. 6,872,386 B2, ORAL VACCINES. Mar. 29, 2005 and U.S. Pat. No. 7,807,144 B2 Oct. 5, 2010

In the invention, the nucleic acid encoding the antigen that triggers the immune response, is cloned into a recombinant vector. The nucleic acid encoding the antigen is operatively linked to one or more regulatory sequences. The regulatory sequences can be those that direct constitutive expression of the antigen, as well as inducible sequences. The recombinant vector can be designed based on such factors for expression in Listonella sp. It may contain more than one nucleic acid encoding different antigens. For example, a recombinant vector contains nucleic acids encoding two antigens, which can induce immune responses against the same or different pathogens. Alternatively, the recombinant vector may contain a nucleic acid encoding a polypeptide (e.g., a helper epitope) that is not antigenic, but itself or its encoded peptide serves to enhance an immune response against a targeted pathogen.

According to the invention, a nucleic acid encoding a viral subunit is constructed in an expression vector, which is operatively linked to a promoter for Listonella sp., particularly, L. anguillarum HP-60 promoter.

Furthermore, the oral vaccine of the invention may be encapsulated in Artemia sp., such as Artemia nauplii. The process for the encapsulation was disclosed in U.S. Pat. Nos. 6,872,386 and 7,807,144 invented by one of the inventors, which are incorporated by reference herein in its entirety. According to the invention, the oral vaccine of the invention is encapsulated in Artemia sp., such as Artemia nauplii, which provides an effective way to provide a solution to the problem due to difficult antigen degradation in the digestive tract of the aquatic animal.

The following examples are offered to illustrate this invention and are not to be construed in any way as limiting the scope of the present invention.

### Example

### 1. Materials and Methods

1.1 Bacteriaum and Fish

L. anguillarum strain TW0302 was isolated from seawater in fish farm at Tainan. All experimental fish were produced from the indoor NNV-free hatchery and were NNV free as confirmed by RT-PCR. All challenge trails were carried out in our indoor NNV-free fish experimental facility.

1.2 Construction of pgroEp-NNVcp Fusion Plasmid

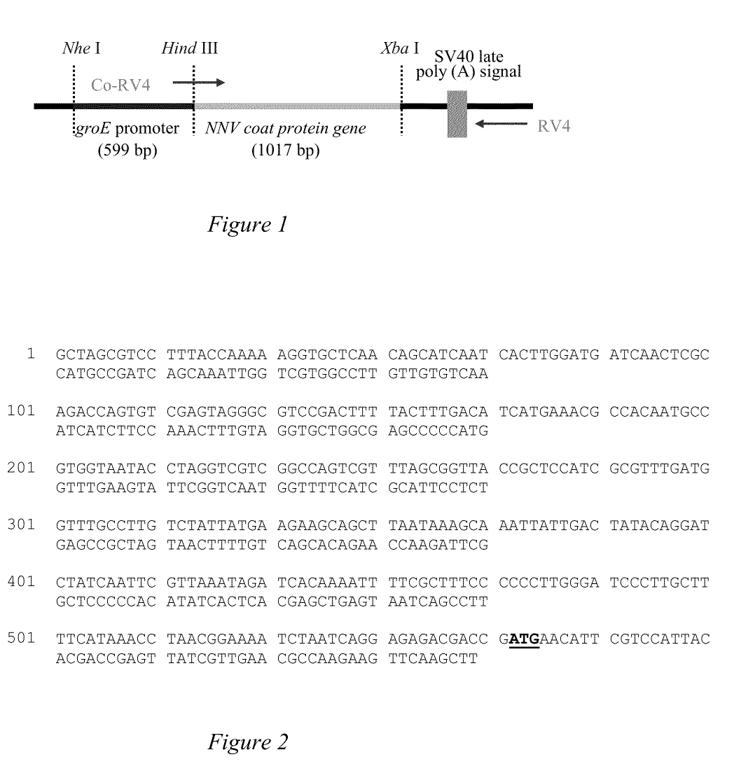

A p groEp-NNVcp (NNV coat protein) fusion plasmid based on the pGL3 plasmid (as shown in FIG. 1) was constructed. NNVcp gene was cloned from an NNV strain isolated from an infected E. coioides [Lin C-C et al., An oral nervous necrosis virus vaccine that induces protective immunity in larvae of grouper (Epinephelus coioides). Aquaculture 268:265-273, 2007]. The pgroE promoter (heat shock protein 60 was isolated by PCR amplification of the nucleotides from −541 to +58 that coding the translation initiation site of the heat shock protein 60 gene groES in L. anguillarum. The temperature cycles of the PCR started with a initial denaturation at 94° C. for 5 min, followed by 30 cycles of 94° C. for 30 sec, 60° C. for 50 sec, and 70° C. for 60 sec, and a final extension reaction at 72° C. for 6 min. The sequence of the isolated promoter was given in FIG. 2DNA sequence of groE promoter region from nucleotide numbers −541 to +58 relative to the translation initiation site (ATG underlined).

1.3 Construction of Shuttle pgroEp-NNVcp Plasmid and Transformation to L. anguillarum

The expression hosts used to produce recombinant NNVcp were L. anguillarum strain TW0302 isolated in our laboratory or E. coli strain BL21 (DE3). Recombinant pgroEp-NNVcp plasmid was transformed to L. anguillarum by electroporation [Cutrin J M et al., Genetic transformation of Vibrio anguillarum and Pasteurella piscicida by electroporation. FEMS Microbiol Lett 128:75-80, 1995], then selected for the ampicillin resistant trait using a TSB agar plate supplemented with 0.1 mg ml⁻¹ ampicillin sodium. The bacterial colonies grown on the plate were further analyzed by PCR using temperature profiles described in section 2.1, with the set of primers listed in Table 1, to verify the presence and ensure the percentage of the NNVcp containing plasmid.

1.4 Analysis of the Optimal Temperature Induction Condition of pgroEp-NNVcp in L. anguillarum

The transformed L. anguillarum was inoculated into a 2 L flask containing 600 ml TSB broth supplemented with 0.1 mg ml⁻¹ sodium ampicillin, cultivated by shaking (200 rpm) at 25° C. until the OD₆₀₀ reached 1.0, then incubated for inducing NNVcp.

1.5 Measurement of Antigen Dosage in Oral Vaccines

The oral vaccines were prepared by encapsulating the inactivated bacteria and the mock versions in Artemia nauplii according to Lin's reference (Aquaculture 268:265-27312007).

In order to determine how number of bacteriim and the quantity of antigen encapsulated in one Artemia, we used a real time PCR method to quantify the number of bacterium, and SDS-PAGE/west blot method to measure the amount of antigen in these two bacterial hosts. A method based on real time PCR technology was designed to determine the amount of L. anguillarum or E. coli encapsulated in each Artemia . Briefly, duplicate samples of 10⁵ to 10⁹ CFU of L. anguillarum or E. coli, and Artemia individuals encapsulated with E. coli or L. anguillarum were collected, washed thoroughly with deionized water, treated according to Prol M J et al. (Real-time PCR detection and quantification of fish probiotic Phaeobacter strain 27-4 and fish pathogenic Vibrio in microalgae, rotifer, Artemia and first feeding turbot (Psetta maxima) larvae. J Appl Microbiol 106:1292-1303, 2009) to obtain purified DNA, and analyzed by real time PCR using the temperature profiles described in Rojo I et al. (Innate immune gene expression in individual zebrafish after Listonella anguillarum inoculation. Fish Shellfish Immunol 23:1285-1293, 2007) with an adequate set of primers designed for E. coli or L. anguillarum (Table 1).

TABLE 1
Primers used for PCR

| Purposes | Primer (5′-3′) |
|---|---|
| Cloning and | Gro_F2 |
| sequencing of pgroE | CTAGCTAGCTCTAAATTTCAT |
| | CATCTGTTCGGCGAG |
| | Gro_R2 |
| | CCCAAGCTTGAACTTCTTGGC |
| | GTTCAACGATAACTCG |
| Confirmation of | RV4 |
| pgroEp-NNVcp fusion | GACGATAGTCATGCCCCGCG |
| plasmid | Co-RV4 |
| transformation | AAGCTTCCATGGTACGCAAAG |
| Determination | |
| of gene | |
| expression levels | |
| 1. Mx gene | Mx_F_rt |
| | AGAAGGTGCGTCCCTGCAT |
| | Mx_R_rt |
| | CTGACAGCGCCTCCAACAC |
| 2. IL-1β | IL-1β_F_rt |
| | CGACATGGTGCGGTTTCTCT |
| | IL-1β_R_rt |
| | CTCTGCTGTGCTGATGTACCAGTT |
| 3. TNF-α | TNF-α_F_rt |
| | CGACAATCAGGCCAAAGAGA |
| | TNF-α_R_rt |
| | AAGCCGCCCTGAGCAAAC |
| 4. β-actin | β-actin_F_rt |
| | GCCCCACCAGAGCGTAAATA |
| | β-actin_R_rt |
| | CATCGTACTCCTGCTTGCTGAT |
| Determination | |
| of bacterial | |
| amount encapsulated | |
| in each Artemia | |
| 1. E. coli | GroE F rt |
| | GCACGAAGAGATTGAGGCAGTTGAG |
| | GroE R rt |
| | GCGCGAATTTGTGGGCTTTTT |
| 2. L. anguillarum | pGroE F rt |
| | GAGCCGCTAGTAACTTTTGTCA |
| | pGroE R rt |
| | GAATGTTCATCGGTCGTCTCTC |

Standard curves relating Ct values to bacterial number (CFU) were obtained by analyzing the PCR measurements with Microsoft Excel software, and quantification of bacteria in each Artemia was conducted by running the same real-time PCR analysis with an adequate set of primers and standard curves.

The SDS-PAGE/western blotting was performed according to Chen Y M et al. (Grouper Mx confers resistance to nodavirus and interacts with coat protein. Dev Comp Immunol. 32:825-836, 2008). The protein extracted from 4×10⁷ CFU recombinant E. coli or L. anguillarum was loaded onto the same gel, and the relative amount of antigen was determined according to the analysis of western blot staining image by using Image J software (NIH, Bethesda, Md., USA).

1.6 Preparation of NNVcp Oral Vaccine Using E. Coli and L. Anguillarum Expression Host

To prepare L. anguillarum based oral vaccine, the cells with maximal NNVcp content were washed by centrifugation (2,500×g, 15 min), rinsed with 600 ml sterile seawater, re-suspended in sterile seawater at 1×10¹⁰ CFU ml⁻¹, deactivated at 65° C. in a water bath for 10 min, and cooled on ice for 10 min to obtain the subunit vaccine product. A L. anguillarum mock subunit vaccine was also produced by using non-transformed L. anguillarum following the same procedures.

To prepare an E. coli-based NNV subunit vaccine, the same p groEp-NNVcp fusion plasmid was transformed into E. coli strain BL21 (DE3), and the transformed E. coli cells were harvested and deactivated by the same procedures for the preparation of the L. anguillarum-based subunit vaccine, with the exception of the use of sterile PBS buffer (137 mM NaCl; 2.7 mM KCl; 10 mM Na₂HPO_{4;} 2 mM KH₂PO₄; adjusted to pH 7.4 with 6 N HCl) instead of sterile seawater for rinsing and suspending cells. A mock subunit vaccine was prepared by using non-transformed E. coli following the same protocol used for preparing E. coli-based subunit vaccine.

1.7 Immunization of Larvae

Thirty days post-hatch (dph) larvae of E. coioides, which were confirmed free of NNV by RT-PCR, were used in the study. Five hundred larvae were randomly divided into five groups (n=100) and immunized. One group was a placebo group fed Artemia only, the other four groups were fed with Artemia encapsulated mock) of E. coli based oral vaccines, and two parallel groups fed with mock (transformed with pgroEp,) and NNVcp subunit vaccine produced in L. anguillarum (transformed with pgroEp-NNVcp). Fish were kept in five separate 20-L tanks with aeration with temperature maintained at 26±1° C. and larvae were acclimated for 3 days before immunization. A single dose of four individual oral vaccines or placebo was given 4 times at 33.0, 33.5, 34.0, and 34.5 dph. After immunization all groupers were fed with untreated Artemia twice a day until the end of the trial.

1.8 Assay the Expression of Innate Immunity Related Genes by RT-PCR

To evaluate the immune-stimulant effects of the subunit vaccine expressed in L. anguillarum, 3 grouper larvae were randomly selected from experimental groups fed with mock E. coli or L. anguillarum from 0 to 96 h after immunization (from 34.5 to 38.5 dph), killed with an overdose of anesthetic, and dissected to remove the brain and viscera.

RNAs were isolated and subjected to real time PCR to measure their expression levels of the three innate-immunity related genes i.e. IL-1β, TNF-α, or Mx isolated and sequenced from E. coioides in our laboratory. The total RNA was extracted from these samples, cDNA was synthesized, and real-time PCR was performed, wherein the set of primers used are given in Table 1. The significant of difference of expression of immune genes were statistically analyzed by one-way ANOVA and Duncan's new multiple range test.

1.9 Measurement of NNVcp Antibody

At 40 dph, 1 week after the beginning of oral vaccination, three fish were sampled randomly from each group, killed, homogenized, and centrifuged to obtain humoral extracts, and analyzed by ELISA to determine anti-NNVcp antibody titer. The ELISA analysis was performed, and the statistical analysis of antibody titer was proceeded by using one-way ANOVA and Dunnett's tests.

1.10 Challenge

NNV was used for challenging trail. At 40 dph, 40 fry were taken randomly from each group and injected intraperitoneally with 10 μl of virus solution with TCID₅₀ as 1.0×10^{5.5}, pre-determinded as a lethal dosage. The mortality of grouper fry in each group was recorded daily for 3 weeks post-challenge, and the final cumulative mortality rates were used to calculate relative percent survival (RPS).

### 2 Results

2.1 Construction of pgroEp-NNVcp Fusion Plasmid

The NNVcp gene was cloned under the control of pgroEp promoter isolated from L. anguillarum and put on a shuttle vector pGL 3, The genetic construct, the promoter region of recognition sites between two PCR primers—RV4 and Co-RV4 and the resulted pgroEp-NNVcp plasmid were illustrated in FIG. 1. The primers used are listed in Table 1 and the DNA sequence of pgroE obtained is listed in FIG. 2.

2.2 The Optimal Temperature Induction Condition of pgroEp-NNVcp in L. anguillarum

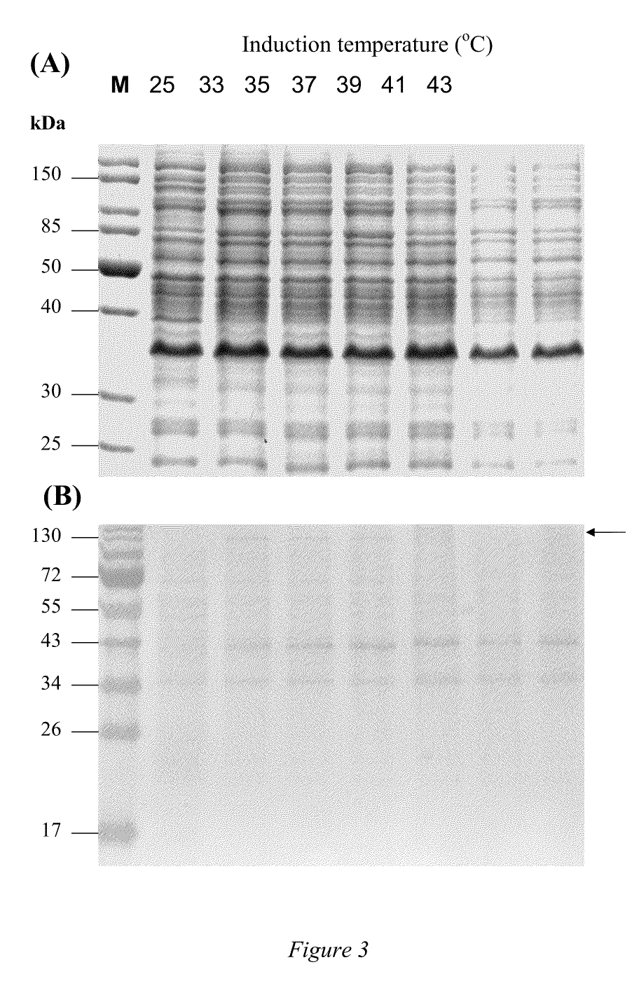

It was found that the best induction temperature for NNVcp production was 37° C. The recombinant L. anguillarum was cultivated at 25° C. then shift to 37° C. to induce the synthesis of NNVcp. The heat induction resulted in significant NNVcp production in recombinant L. anguillarum as shown in FIG. 3.

2.3 Measurement of the Number of Bacterium in Each Artemia

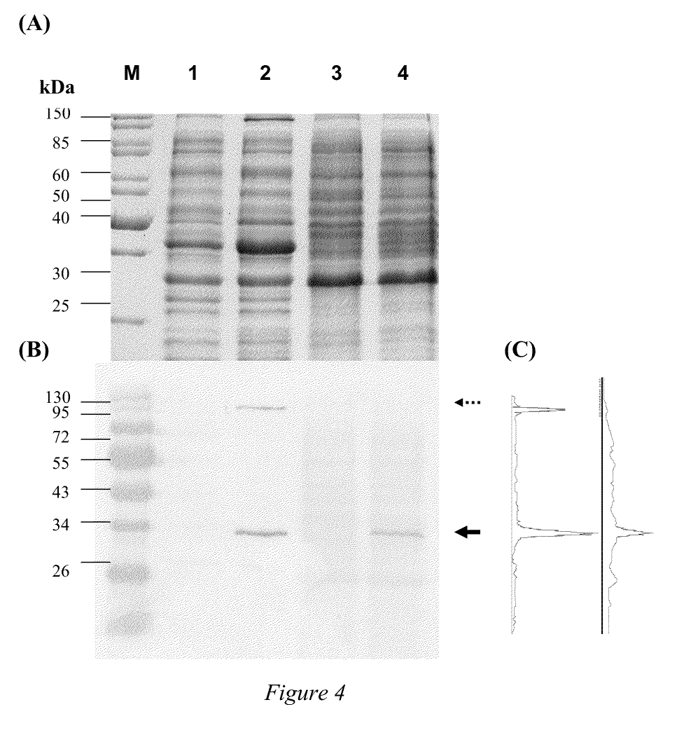

As bacterium once eaten bt Artemia, it could be rapidly digested, so that the conventional bacterial colony counting method to measure the number of bacteraium encapsulated into Artemia could be not accurate. The number of bacterium in each Artemia was then measured by assaying the copy of bacterial gene in Artemia using RT-PCR. The relationship between Ct value and bacteria number can be fit well with equation obtained from non-linear regression (FIG. 4). According to the formulas obtained from the regressions, the average number of E. coli and L. anguillarum incorporated in each Artemia was determined as 8.3±4.2×10⁴ and 4.1±2.1×10⁶ CFU, respectively.

2.4 The Determination of NNVcp Antigen in Each Dosage of Oral Vaccines

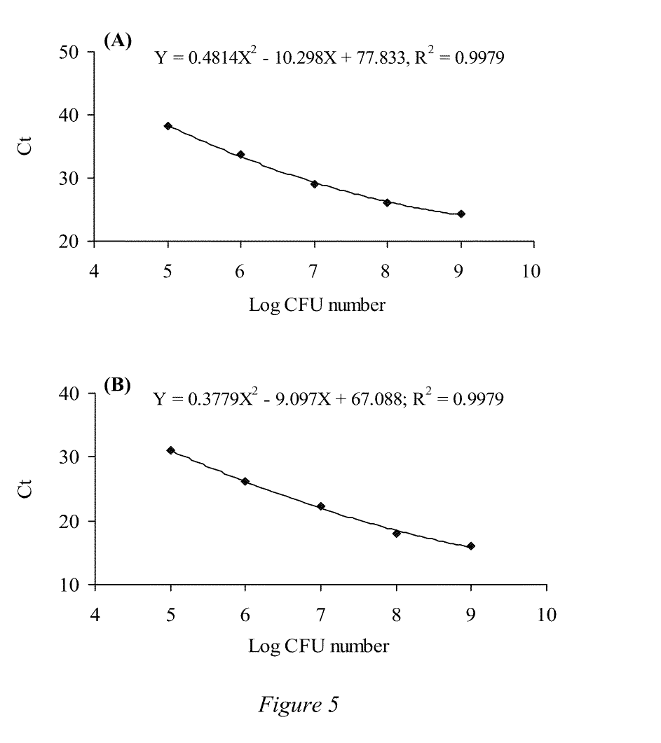

The SDS-PAGE/western blot analysis confirmed the production of NNVcp in the E. coli and L. anguillarum expression hosts (indicated by solid arrow in FIG. 5A). The NNVcp protein band was immunologically confirmed by using monoclonal antibody of NNVcp with higher molecular weight (indicated by dashed arrow in FIG. 5B). An aggregated NNVcp protein usually was observed and gradually dissociated into NNVcp monomer after prolonged incubation with SDS buffer (data not shown). This aggregated NNVcp accounted for approximate ⅓ of the total NNVcp produced in recombinant E. coli, but much less in L. anguillarum expression system estimated according to the analytical result of Image J (FIG. 5C). According to the result showed in FIG. 5C, the amount of NNVcp produced in L. anguillarum is only ⅕ of that of the E. coli on the same CFU basis. Since the average amount of L. anguillarum encapsulated in one Artemia was 5 times of the amount of E. coli incorporated, and for the same CFU number of bacteria, the amount of NNVcp in L. anguillarum contain ⅕ that in E. coli, the Artemia encapsulated with either recombinant L. anguillarum or E. coli should have the same amount of NNVcp antigen.

2.5 Innate-Immunity Stimulation Effects Related to Expression Hosts

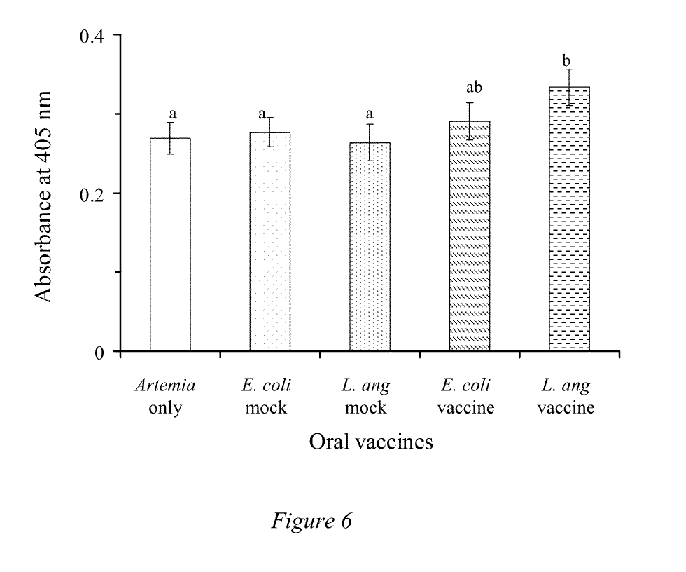

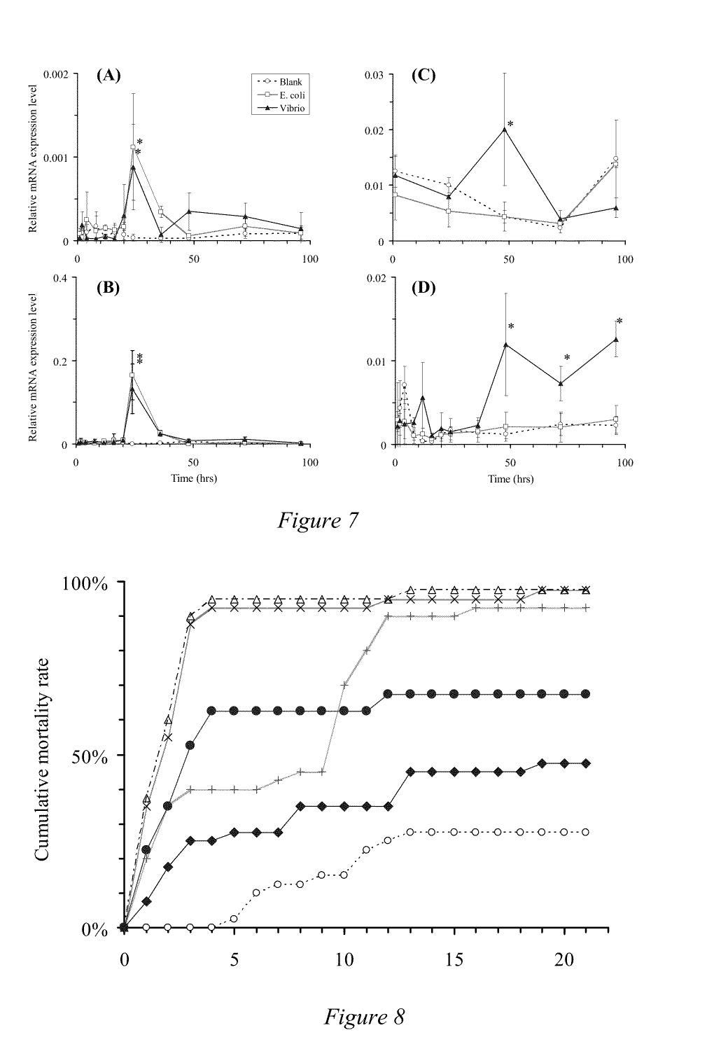

The intake of both mock E. coli and L. anguillarum significantly upregulated TNF-α and IL-1β genes in viscera of grouper fry. The transcription levels of the two genes peaked at 24 h post-vaccination and then declined immediately (FIG. 6). However, the ingestion of L. anguillarum based oral vaccines (groupe X and Y) significantly increased the expression of Mx in brain and viscera of grouper. The transcriptional level of Mx gene in both brain and viscera rose, peaking 48 h after consumption (P<0.05) (FIG. 6). In contrast, the E. coli based oral vaccines (group W and Z) failed to induce significant amount of Mx expression in either viscera or brain.

As known in fish oral vaccine, no considerable amount of NNVcp serum specific antibody was found after the group immunized with E. coli based oral vaccine. However, it was found that L. anguillarum-based oral vaccine significantly elicited antigen-specific antibody level at 7 days post-vaccination (P<0.05) (FIG. 8).

2.7 Challenge Test

Fish challenged with NNV died from 1-18 days post-challenge, with maximal mortality occurring during the first week in all groups (FIG. 8). The mock L. anguillarum provided better protection than the E. coli-based subunit vaccines for the first 9 days. The final mortality rate was 27.5% for the placebo group challenged with L15 medium only, and on vaccinated groups, 97.5% in placebo and mock E. coli groups, 92.5% in the mock L. anguillarum group, 67.5% in E. coli-based vaccine group, and 47.5% in L. anguillarum-based vaccine group. Based on final mortality rates, the RPS of the two vaccines were 30.8% for the E. coli-based vaccine, and 51.3% for the L. anguillarum-based vaccine.

In conclusion, the protective efficacy for aquatic animals could be improved by using L. anguillarum as an expression host with an improved immunogenicity.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

I/We claim:

1. An oral vaccine with an enhanced protective efficacy for aquatic animals comprising an antigen, which is expressed in Listonella sp. as an expression host transformed with an expression vector comprising the nucleic acid encoding the antigen.

2. The oral vaccine of claim 1, wherein the protective efficacy is prophylactic.

3. The oral vaccine of claim 1, wherein the protective efficacy is therapeitic.

4. The oral vaccine of claim 1, wherein the antigen is expressed in L. anguillarum as an expression host transformed with an expression vector comprising the nucleic acid encoding the antigen.

5. The oral vaccine of claim 1, wherein the nucleic acid encoding the antigen is operatively linked to a promoter that is functional in Listonella sp.

6. The oral vaccine of claim 4, wherein the nucleic acid encoding the antigen is operatively linked to L. anguillarum HP-60 promoter.

7. The oral vaccine of claim 1, wherein the antigen is a subunit protein.

8. The oral vaccine of claim 1, wherein the antigen is a viral subunit.

9. The oral vaccine of claim 7, wherein the antigen is a subunit of Nervous Necrosis Virus (NNV).

10. The oral vaccine of claim 1, which is encapsulated in Artemia sp.

11. The oral vaccine of claim 9, which is encapsulated in Artemia nauplii.

12. A method for preparing an oral vaccine with an enhanced protective efficacy for aquatic animals comprising expressing an antigen in Listonella sp. as an expression host transformed with an expression vector comprising the nucleic acid encoding the antigen, and encapsulating the antigen as expressed in Artemia sp.

13. The method of claim 11, wherein the antigen is expressed in L. anguillarum as an expression host.

14. The method of claim 11, wherein the nucleic acid encoding the antigen is operatively linked to a promoter functional in Listonella sp.

15. The method of claim 12, wherein the nucleic acid encoding the antigen is operatively linked to L. anguillarum HP-60 promoter.

16. The oral vaccine of claim 1, wherein the antigen is a subunit protein.

17. The method of claim 11, wherein the antigen is a viral subunit.

18. The method of claim 15, wherein the antigen is a subunit of Nervous Necrosis Virus (NNV).

19. The method of claim 11, wherein the antigen as expressed is encapsulated in Artemia nauplii.


SEQUENCE LISTING (17 sequences)

SEQ ID NO: 1 - DNA, 36 bp, Artificial Sequence
  Gro_F2
ctagctagctctaaatttcatcatctgttcggcgag

SEQ ID NO: 2 - DNA, 37 bp, Artificial Sequence
  Gro_R2
cccaagcttgaacttcttggcgttcaacgataactcg

SEQ ID NO: 3 - DNA, 20 bp, Artificial Sequence
  RV4
gacgatagtcatgccccgcg

SEQ ID NO: 4 - DNA, 21 bp, Artificial Sequence
  Co-RV4
aagcttccatggtacgcaaag

SEQ ID NO: 5 - DNA, 19 bp, Artificial Sequence
  Mx_F_rt
agaaggtgcgtccctgcat

SEQ ID NO: 6 - DNA, 19 bp, Artificial Sequence
  Mx_R_rt
ctgacagcgcctccaacac

SEQ ID NO: 7 - DNA, 20 bp, Artificial Sequence
  IL-1beta_F_rt
cgacatggtgcggtttctct

SEQ ID NO: 8 - DNA, 24 bp, Artificial Sequence
  IL-1 beta_R_rt
ctctgctgtgctgatgtaccagtt

SEQ ID NO: 9 - DNA, 20 bp, Artificial Sequence
  TNF-alpha_F_rt
cgacaatcaggccaaagaga

SEQ ID NO: 10 - DNA, 18 bp, Artificial Sequence
  TNF-alpha_R_rt
aagccgccctgagcaaac

SEQ ID NO: 11 - DNA, 20 bp, Artificial Sequence
  beta-actin _F_rt
gccccaccagagcgtaaata

SEQ ID NO: 12 - DNA, 22 bp, Artificial Sequence
  beta-actin _R_rt
catcgtactcctgcttgctgat

SEQ ID NO: 13 - DNA, 25 bp, Artificial Sequence
  GroE F rt
gcacgaagagattgaggcagttgag

SEQ ID NO: 14 - DNA, 21 bp, Artificial Sequence
  GroE R rt
gcgcgaatttgtgggcttttt

SEQ ID NO: 15 - DNA, 22 bp, Artificial Sequence
  pGroE F rt
gagccgctagtaacttttgtca

SEQ ID NO: 16 - DNA, 22 bp, Artificial Sequence
  pGroE R rt
gaatgttcatcggtcgtctctc

SEQ ID NO: 17 - DNA, 599 bp, Listonella anguillarum
gctagcgtcctttaccaaaaaggtgctcaacagcatcaatcacttggatgatcaactcgccatgccgatcagcaaattggtcgtggccttgttgtgtcaaagaccagtgtcgagtagggcgtccgacttttactttgacatcatgaaacgccacaatgccatcatcttccaaactttgtaggtgctggcgagcccccatggtggtaatacctaggtcgtcggccagtcgtttagcggttaccgctccatcgcgtttgatggtttgaagtattcggtcaatggttttcatcgcattcctctgtttgccttgtctattatgaagaagcagcttaataaagcaaattattgactatacaggatgagccgctagtaacttttgtcagcacagaaccaagattcgctatcaattcgttaaatagatcacaaaattttcgctttccccccttgggatcccttgcttgctcccccacatatcactcacgagctgagtaatcagccttttcataaacctaacggaaaatctaatcaggagagacgaccgatgaacattcgtccattacacgaccgagttatcgttgaacgccaagaagttcaagctt